(19) United States
(12) Patent Application Publication
(10) Pub. No.: US 2023/0187033 A1
(43) Pub. Date: Jun. 15, 2023

(54) METHOD FOR PRESCRIBING A CLINICAL TRIAL

(71) Applicant: PIPA LLC, Davis, CA (US)

(72) Inventors: Ilias Tagkopoulos, Davis, CA (US); Minseung Kim, Davis, CA (US)

(21) Appl. No.: 18/105,617

(22) Filed: Feb. 3, 2023

Related U.S. Application Data
(63) Continuation-in-part of application No. 17/987,535, filed on Nov. 15, 2022.
(60) Provisional application No. 63/306,456, filed on Feb. 3, 2022.
(60) Provisional application No. 63/280,532, filed on Nov. 17, 2021.

(51) Int. Cl. G16H 10/20; G16H 10/60; G06F 40/40

(52) U.S. Cl. CPC G16H 10/20; G16H 10/60; G06F 40/40

(57) ABSTRACT

A method includes: receiving a query for a target health condition, a target compound from a research portal; identifying a set of edges coupling a target node representing the target health condition and a termination node representing the target compound in a semantic network, each edge in the set of edges includes intermediate nodes representing a set of patient characteristics and a composite action characteristic. The method also includes, in response to a first composite action characteristic falling within a first range and in response to a second composite action characteristic falling within a second range: isolating a first patient characteristic, contained in a first set of patient characteristics, as inclusion criteria; and defining a second patient characteristic, contained in a second set of patient characteristics, as exclusion criteria; and aggregating inclusion criteria and exclusion criteria into a specification for a clinical trial.

### CROSS-REFERENCE TO RELATED APPLICATIONS

This Application claims the benefit of U.S. Provisional Application No. 63/306,456, filed on 3 Feb. 2022, which is incorporated in its entirety by this reference.

This Application is also a continuation-in-part of U.S. patent application Ser. No. 17/987,535, filed on 15 Nov. 2022, which claims the benefit of U.S. Provisional Application No. 63/280,532, filed on 17 Nov. 2021, each of which is incorporated in its entirety by this reference.

### TECHNICAL FIELD

This invention relates generally to the field of bioinformatics and data science and more specifically to a new and useful method for prescribing a clinical trial in the field of bioinformatics and data science.

### BRIEF DESCRIPTION OF THE FIGURES

FIGS. 1A and 1B are a flowchart representation of a method;

FIGS. 2A and 2B are a flowchart representation of one variation of the method; and

FIGS. 3A and 3B are a flowchart representation of one variation of the method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

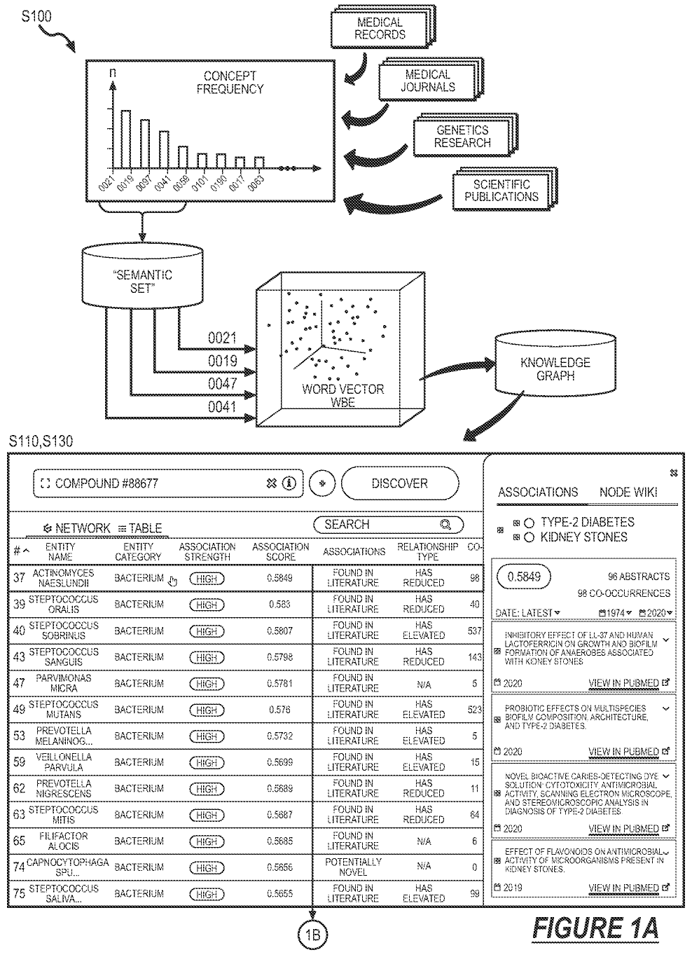

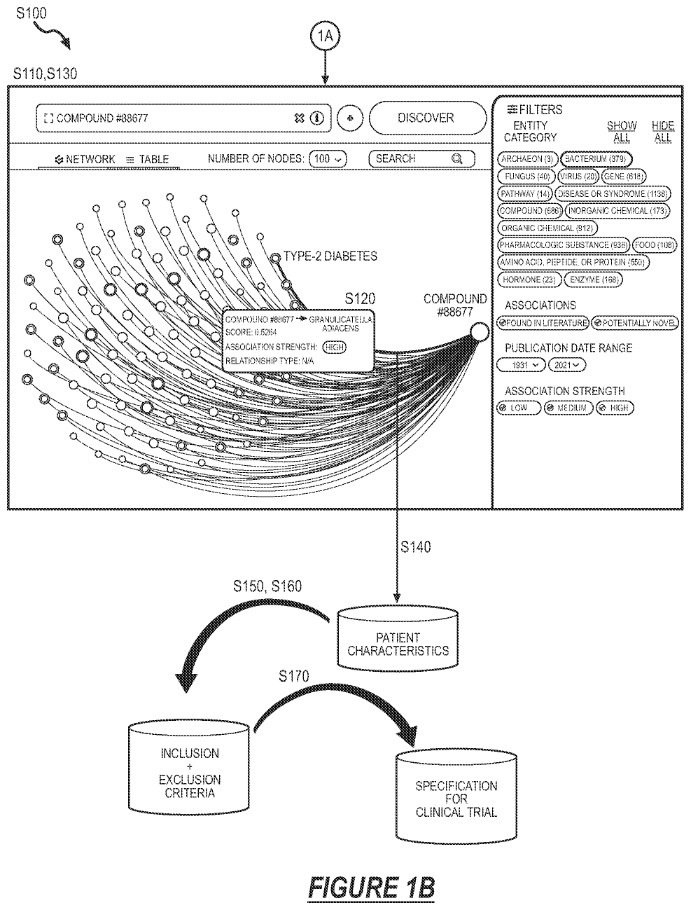

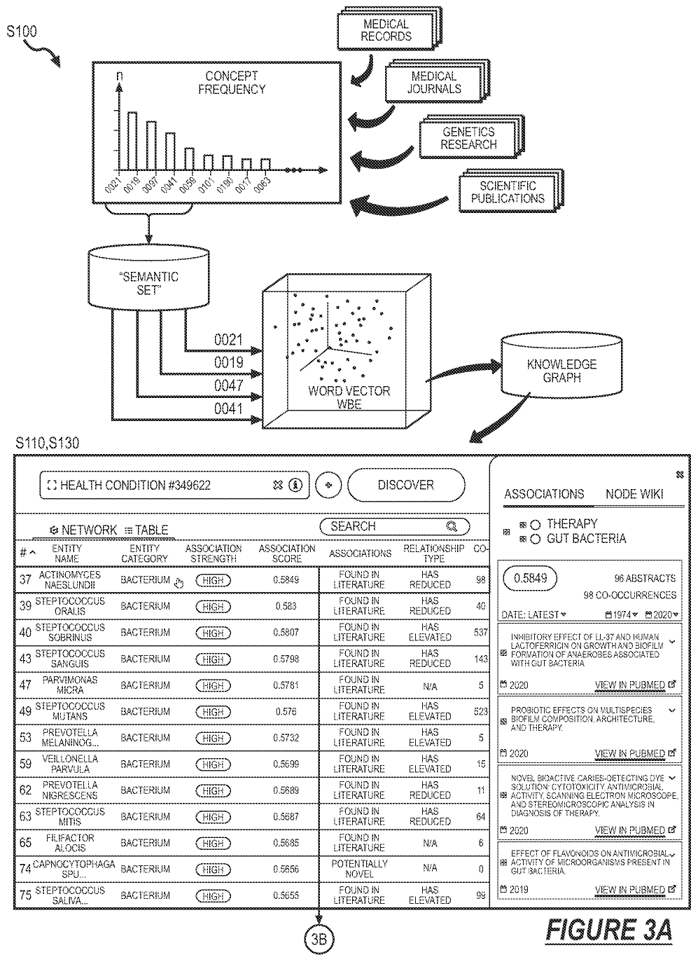

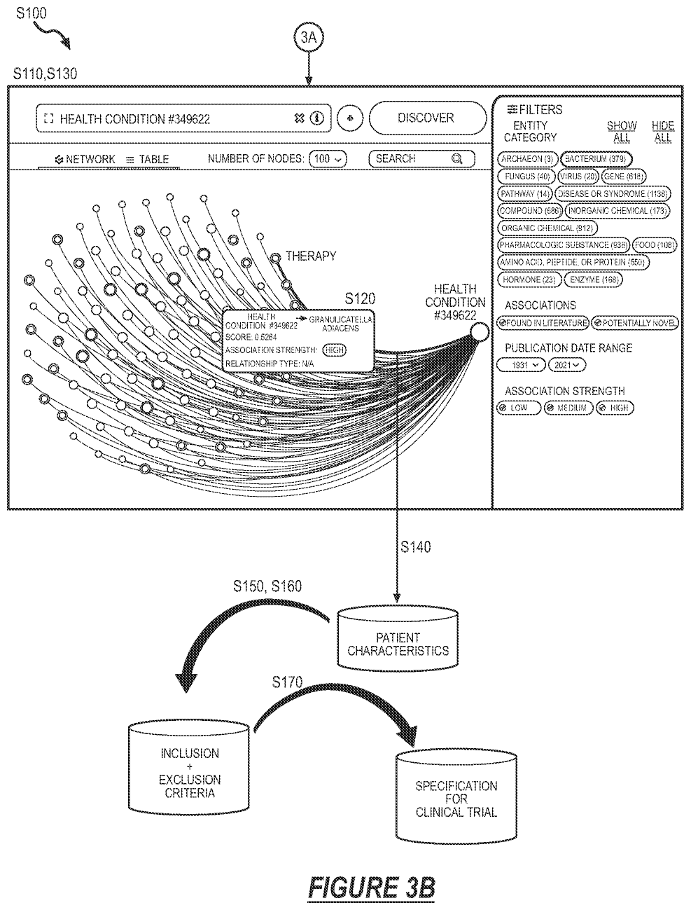

As shown in FIGS. 1A, 1B, 2A, 2B, 3A, and 3B, a method S100 for prescribing a clinical trial includes: receiving a query for a target compound, a target health condition, and a target direction from a research portal in Block S110; identifying a first edge coupling a target node representing the target compound and a termination node representing the target health condition in a semantic network in Block S120; calculating a first composite action characteristic for the first edge based on a first combination of action characteristics stored in connections along the first edge in Block S130; extracting a first set of patient characteristics from intermediate nodes, storing chemical and biological concepts, along the first edge in Block S140; identifying a second edge coupling the target node representing the target compound and the termination node representing the target health condition in the semantic network in Block S120; calculating a second composite action characteristic for the second edge based on a second combination of action characteristics stored in connections along the second edge in Block S130; and extracting a second set of patient characteristics from intermediate nodes, storing chemical and biological concepts, along the second edge in Block S140. The method S100 also includes: in response to the first composite action characteristic falling within a first range corresponding to the target direction and, in response to the second composite action characteristic falling within a second range distinct from the target direction, defining a first patient characteristic, contained in the first set of patient characteristics and excluded from the second set of patient characteristics, as inclusion criteria in Block S150 and defining a second patient characteristic, contained in the second set of patient characteristics and excluded from first set of patient characteristics, as exclusion criteria in Block S160. The method S100 also includes aggregating inclusion criteria and exclusion criteria into a specification for the clinical trial in Block S170 .

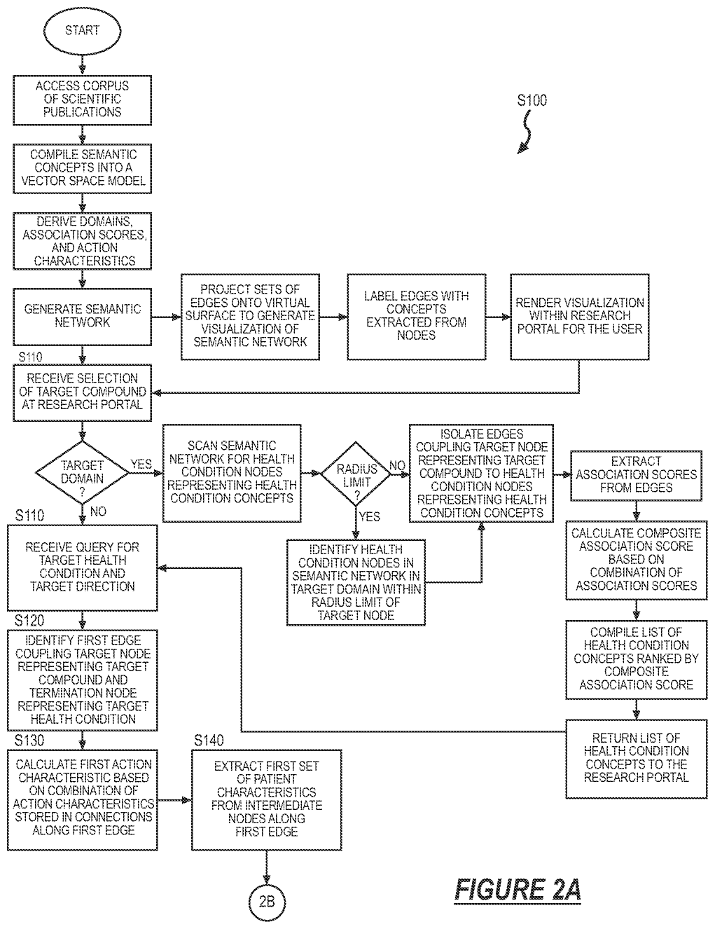

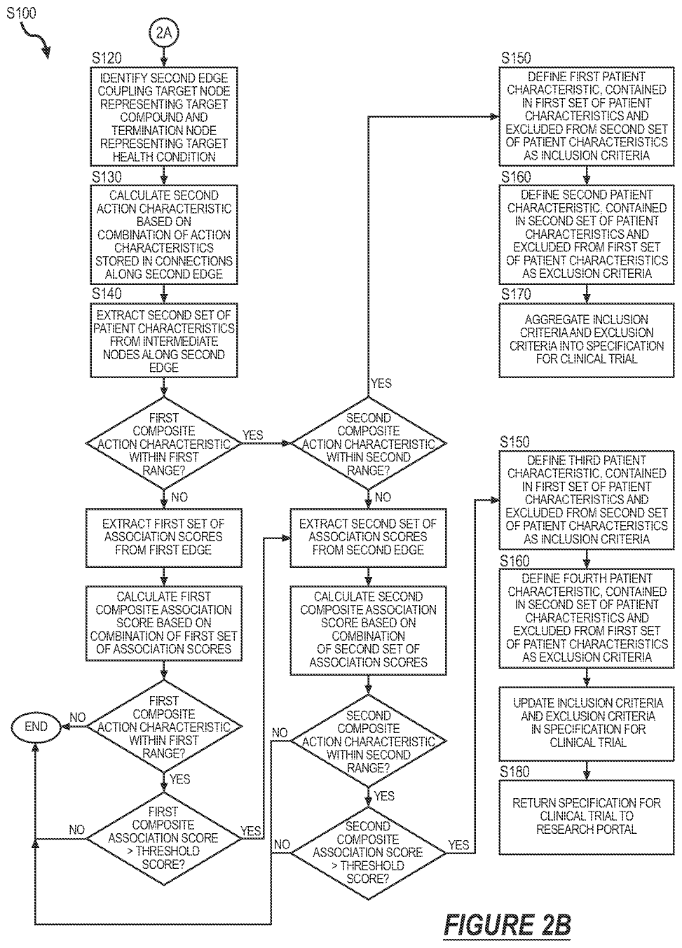

As shown in FIGS. 2A and 2B, one variation of the method S100 includes: receiving a query for a target compound, a target health condition, and a target direction from a research portal in Block S110; identifying a first edge coupling a target node representing the target compound and a termination node representing the target health condition in a semantic network in Block S120; extracting a first composite action characteristic and a first composite association score from connections along the first edge in Block S130; extracting a first set of patient characteristics from intermediate nodes, storing chemical and biological concepts, along the first edge in Block S140; identifying a second edge coupling the target node representing the target compound and the termination node representing the target health condition in the semantic network in Block S120; extracting a second composite action characteristic and a second composite association score from connections along the second edge in Block S130; and extracting a second set of patient characteristics from intermediate nodes, storing chemical and biological concepts, along the second edge in Block S140. This variation of the method S100 further includes: in response to the first composite action characteristic falling within a first range corresponding to the target direction and, in response to the first composite association score exceeding a threshold score, defining a first patient characteristic, contained in the first set of patient characteristics and excluded from the second set of patient characteristics, as inclusion criteria in Block S150; in response to the second composite action characteristic falling within a second range distinct from the target direction and in response to the second composite association score exceeding the threshold score, defining a second patient characteristic, contained in the second set of patient characteristics and excluded from first set of patient characteristics, as exclusion criteria in Block S160; aggregating inclusion criteria and exclusion criteria into a specification for a clinical trial in Block S170; and returning the specification for the clinical trial to the research portal in Block S180.

One variation of the method S100 includes: receiving a query for a target health condition, a target compound and a target direction from a research portal in Block S110; identifying a first edge coupling a target node representing the target health condition and a termination node representing the target compound in the semantic network, the first edge including intermediate nodes representing a first set of patient characteristics and a first composite action characteristic in Block S120; and identifying a second edge coupling the target node and a termination node in the semantic network, the second edge including intermediate nodes representing a second set of patient characteristics and a second composite action characteristic in Block S120. This variation of the method S100 further includes, in response to the first composite action characteristic falling within a first range corresponding to the target direction and, in response to the second composite action characteristic falling within a second range distinct from the target direction: isolating a first patient characteristic, contained in the first set of patient characteristics and excluded from the second set of patient characteristics, as inclusion criteria in Block S150; and defining a second patient characteristic, contained in the second set of patient characteristics and excluded from first set of patient characteristics, as exclusion criteria in Block S160. This variation of the method S100 also includes aggregating the inclusion criteria and the exclusion criteria into a specification for the clinical trial in Block S170 .

### 2. Applications

Generally, the method can be executed by a computer system (e.g., a computer network, a remote computer system) to: derive associations between language concepts (e.g., chemical compounds, genes, diseases, microbes) based on proximities of these concepts across a corpus of resources (e.g., scientific publications, medical records); derive connections between associated language concepts based on action descriptors in the corpus of resources; derive domains or concept types of these language concepts based on domain descriptors in the corpus of resources; and represent these language concepts, the strengths and connections between these language concepts, and the domains of these language concepts in a semantic network (e.g., knowledge graph, ontology).

The computer system can further execute Blocks of the method S100 to: receive search terms (e.g., a disease or health condition, a compound or therapy type, a set of patient characteristics) from a user via a user portal (or “research portal”); query the semantic network for edges (e.g., combinations of nodes and nodal connections) that connect nodes (e.g., target node, termination node) that represent these search terms; interface with the user to select a single edge that represents an action pathway of a therapy (hereinafter a “compound”) effecting a health condition (e.g., a disease or a symptom) within a particular subset of a patient population with a particular patient characteristic; isolate a patient characteristic within this particular set of patient characteristics to define inclusion and exclusion criteria; and aggregate these inclusion and exclusion criteria into a specification for a clinical trial to validate efficacy of the therapy in treating the health condition in a particular subset of the patient population exhibiting this patient characteristic.

In particular, the computer system can execute Blocks of the method S100: to generate an effect (e.g., hypothesis) for a mechanism of action for a therapy within a patient with a specific set of patient characteristics—such as a specific combination of genes, gastrointestinal bacteria, and other microbiomes—based on omics data extracted from a large corpus of health records and research resources; to transform these patient characteristics into inclusion and exclusion criteria; and to aggregate these inclusion and exclusion criteria into a specification for a clinical trial to validate the therapy for patients with these patient characteristics.

For example, a user may hypothesize that a compound may be effective in reducing symptoms for patients with Type-2 diabetes and manually set inclusion and exclusion criteria for a clinical trial for this compound based on diabetes diagnosis, age bracket, and geographic region. However, the effect (i.e., the “mechanism of action” or “action pathway”) of this compound within subsets of a patient population may differ—and therefore yield mixed results—due to different genetics, microbiomes, and other biomarkers even though all patients in this population meet basic medical condition, location, and age criteria.

Conversely, the computer system can: predict multiple distinct action pathways for the compound within a patient population based on microbiomes and other patient characteristics stored along edges between nodes in a semantic network that represent the compound and Type-2 diabetes; present these distinct action pathways to the user, such as in the form of a visualization of the semantic network; interface with the user to isolate a particular action pathway from this set; convert characteristics of the particular action pathway into inclusion and exclusion criteria, such as including specific patient microbiomes contained along edges of the semantic network representing these action pathways; and compile these inclusion and exclusion criteria into a specification for a clinical trial to validate efficacy of the compound according to the target action pathway within a particular patient subpopulation with these specific patient microbiomes.

Thus, the computer system can: define inclusion and exclusion criteria that isolate a patient population for which a compound is predicted to effect a health condition according to a particular action pathway; and reduce noise—resulting from different action pathways of the same compound on other patients with the same health condition but different microbiomes—in clinical trial results.

Therefore, the computer system can execute Blocks of the method S100 to streamline research and development of chemical compounds and other therapies for humans (and other animals). For example, the computer system can execute Blocks of the method S100 to identify and propose: new applications of existing compounds to address a target disease; or known applications of existing compounds (and/or microbes, genes, gene therapies, etc.) to address a target disease through novel connections (e.g., edges, action pathways) between associated concepts.

In particular, the computer system: compiles many (e.g., millions) journals, scientific publications, medical records, gene sequences, blood panels, microbiome panels, and/or resources; automatically derives domains, strengths of associations and directions of action pathways between many chemical and biological concepts described across these resources—such as in titles, abstracts, bodies, and/or footnotes of these resources; and represents the chemical and biological concepts, strengths of associations, and directions of action pathways in edges within a semantic network. Accordingly, the computer system can return immediate and meaningful hypotheses for targeted research and development of therapies given minimal search terms, such as merely: a single disease descriptor and a therapy type (e.g., chemical compound or medical treatment); or a single disease descriptor and a pathway type (e.g., bacteria, gene); or a single target concept (e.g., compound, therapy), a target domain (e.g., disease, symptom), and a target direction (e.g., directional keyword).

### 3. Terms

Generally, the semantic network (e.g., knowledge graph, ontology) includes nodes representing biological and chemical concepts labeled with domains and connections between nodes storing association scores and action characteristics.

More specifically, a biological and chemical concept (e.g., a gene sequence, a disease, a microbe, a bioactive compound, a taste quality, a food product) can be represented in nodes containing biological and chemical concepts. Domains in the semantic network can include health conditions, diseases, compounds, symptoms, therapies, genes, bacterium, microbiomes, etc. Association scores can be stored in connections between nodes along edges in the semantic network and represent strengths of correlations between two concepts based on proximity in the word vector cube and/or based on proximity of these two concepts in individual resources across the corpus of resources. Furthermore, composite association scores represent the average of association scores from a start node to a terminal node or the average intermediate association scores from a start node to an intermediate node or the average intermediate association scores from an intermediate node to a terminal node.

Similarly, action characteristics represent directions of correlations between connected chemical and biological concepts based on the presence of directional keywords between connected biological and chemical concepts within individual scientific publications of the corpus of scientific publications. More specifically, directional keywords can be divided into two categories: positive actions (e.g., upregulates, catalyzes, starts, causes, promotes, grows, induces, yields) and negative actions (e.g., downregulates, inhibits, stops, prevents, demotes, kills, reduces, suppresses).

Furthermore, a user can enter queries within a user portal (or “research portal”) to inform clinical research (e.g., prescribe a clinical trial) that addresses a target concept and a target domain within the semantic network.

### 4. Resources

Generally, the computer system can access a corpus of resources (e.g., scientific publications) and compile a population of semantic concepts represented in the corpus of resources into a vector space model based on proximity of semantic concepts within individual resources, in the corpus of resources and frequency of semantic concepts across the corpus of resources.

In particular, the computer system can retrieve scientific papers, journal publications, scientific publications, (anonymized) patient health records, genetic data, microbiome data, and/or medical histories, etc. from one or more resource databases.

### 5. Word Vector Cube

Generally, the computer system can construct a vector space model (e.g., a “word vector cube”) that represents (or “embeds”) word representations from the corpus of resources in a continuous vector space where semantically-related word representations are mapped to nearby points in the vector space—that is, semantically-related word representations are “embedded” nearby each other in the vector space.

More specifically, the computer system can generate a multi-dimensional word vector cube that contains a large population of chemical and biological concepts mapped according to semantic proximity derived from the corpus of resources. Each object in the word vector cube: can include a word or phrase representing a chemical or biological concept (e.g., a gene sequence, a disease, a microbe); and can be located at a “distance” (e.g., a multi-dimensional spatial distance, a weight, a proximity value) to another object in the word vector cube corresponding to a frequency that words or phrases represented by these two objects occur together in individual resources in the corpus.

### 5.2 Vector Space Modeling

In one implementation, the computer system: accesses documents from a corpus of resources; detects and discards stop words (e.g., ‘a’, ‘the’, ‘ourselves’, ‘hers’, ‘between’, ‘yourself’, ‘but’, ‘again’, ‘there’, ‘about’, ‘once’, ‘out’) from each document; and initiates generation of the word vector cube based on the remaining words in these documents. The computer system can then implement statistical methods to identify a unique combination of words occurring in each document in this corpus of resources, such as a unique combination of five words or a quantity of words proportional to a length of a document. For example, to identify a unique combination of words in one document in the corpus of resources, the remote computer system can: detect and remove all stop words from the document; convert all plurals of words in the document to their singular forms; implement statistical methods to identify a target quantity of words occurring with greatest frequency in the document; and store these words as a combination of words tagged with a topic label extracted from this document. The remote computer system can repeat this process for each other document in the corpus of resources to generate a population of topic words tagged with topics represented across the corpus of resources.

The computer system can then implement vector space modeling techniques to aggregate this population of objects into a multi-dimensional word vector cube with many nodes—each containing one object in the population—related spatially based on proximity of corresponding topic words occurring throughout the corpus of resources.

### 5.3 Concepts

Generally, the corpus of resources may describe a range of concepts (and directly or indirectly inform relationships between these concepts) in various domains, such as: genes; compounds, pharmacologic substances, inorganic chemicals, and/or organic chemicals; proteins, peptides, and/or amino acids; hormones; enzymes; diseases, syndromes, and/or and disease stages; symptoms and symptom magnitudes; microbes (e.g., bacteria, viruses, fungi); sample population characteristics (e.g., age or age group, gender, geographic location, medical histories, diagnoses, symptoms, treatments, genetic information, blood test results, microbiome panel); treatment or experiment actions (e.g., dose size, administration time windows, administration types); etc.

Accordingly, the computer system can implement the foregoing methods and techniques to extract concepts within these domains from the corpus of resources, to characterize their proximities in these documents and across the corpus of resources, and to represent these proximities within a word vector cube or other vector space model.

### 6. Semantic Network

Generally, the computer system can generate a semantic network that represents proximities (or “associations”) of concepts in the word vector cube, domains of these concepts, and action characteristics (e.g., action directions, correlation direction) between these concepts informed by the corpus of resources.

In one implementation, the computer system can: access a corpus of scientific publications; compile a population of semantic concepts represented in the corpus of scientific publications into a vector space model based on proximity of semantic concepts within individual scientific publications, in the corpus of scientific publications and frequency of semantic concepts across the corpus of scientific publications; derive domains of a set of chemical and biological concepts in the vector space model based on proximity to domain descriptors in the vector space model; derive association scores between connected chemical and biological concepts, in the set of chemical and biological concepts, based on proximity in the vector space model; and derive action characteristics between connected chemical and biological concepts, in the set of chemical and biological concepts, based on action descriptors in the vector space model. The computer system can then generate a semantic network that includes: a set of nodes representing the set of chemical and biological concepts labeled with domains; and a set of edges connecting the set of nodes and storing association scores and action characteristics.

### 6.1 Association Score

In one implementation, the computer system interprets strengths of associations (or “association scores”) between two concepts based on proximity of these concepts within the word vector cube—that is, inversely proportional to an n-dimensional distance between these two concepts in the word vector cube.

In another implement, for two concepts (e.g., two words or two phrases) represented in the word vector cube, the computer system can calculate an association score: proportional to a number of times (or “frequency”) that two concepts appear within the same resource (e.g., within the title, abstract, body, and/or footnotes of the resource); inversely proportional to a distance (e.g., a number of letters or words) between paired instances of these two concepts in the resource; and/or proportional to a number of resources in the corpus of resources that includes at least one instance of each of these two concepts.

Accordingly, the computer system can represent strengths of correlations between two concepts based on proximity in the word vector cube and/or based on proximity of these two concepts in individual resources across the corpus of resources.

### 6.2 Concept Domain

In one implementation, the computer system also predicts domains of concepts represented in the word vector cube and/or filters concepts represented in the word vector cube to include a particular set of relevant (or “target”) domains, such as: genetic information; compounds, pharmacologic substances, inorganic chemicals, and/or organic chemicals; proteins, peptides, and/or amino acids; hormones; enzymes; diseases, syndromes, and/or and disease stages; symptoms; bacteria; viruses; fungi; patient population characteristics; and/or treatment or experiment actions.

For example, the computer system can: apply standard naming conventions for genes or genetic sequences to identify particular words or phrases in the word vector cube as genes and genetic sequences in the semantic network; apply standard naming conventions for compounds and chemical formulae to identify particular words or phrases in the word vector cube as chemical compounds in the semantic network; apply standard naming conventions for diseases and diagnoses to identify particular words or phrases in the word vector cube as diseases in the semantic network; apply standard naming conventions for therapy administration and experiment actions and diagnoses to identify particular words or phrases in the word vector cube as pathway or experiment actions in the semantic network; and label concepts in the semantic network with their domains accordingly

Additionally or alternatively, the computer system can: detect domain descriptors in the word vector cube; and identify or predict the domain of a particular concept (i.e., a word or phrase) in the word vector cube based on a domain descriptor nearest this concept in the word vector cube. For example, the computer system can identify a concept in the word vector cube as “bacterium” if an association score between the concept and other objects—identified as [bacteria, bacterium, organism, prokaryotic, and/or microorganism] domain descriptors in the word vector cube—are high. More specifically, the computer system can identify a concept in the word vector cube as “bacterium” if a combination (e.g., sum) of the association scores between the concept and known bacteria-related language descriptors (e.g., bacteria, bacterium, organism, prokaryotic, and/or microorganism) exceeds a threshold score.

### 6.3 Action Characteristics

Furthermore, the computer system can derive an action characteristic (or “pathogen score”) representing positive or negative correlation between two concepts (e.g., in the same or different domains) based on affirmative and negative language contained in the corpus of resources and/or represented in the word vector cube.

In one implementation, the computer system calculates action characteristics between −1.000 and +1.000. In particular, for two concepts represented in the word vector cube, the computer system can calculate a negative action component: proportional to a number of times (or “frequency”) that the two concepts appear within the same resource with negative language (e.g., “not,” “inhibits”, “down-regulates”, “reverse,” “mitigate,” “reduce,” “attenuate”) surrounding or arranged between these two concepts; inversely proportional to the distance (e.g., number of letters or words) between these two concepts and negative language in the resource; and proportional to a number of resources that includes both concepts with interstitial negative language. The computer system can similarly calculate a positive action component for the two concepts: proportional to a number of times that two concepts appear within the same resource without negative language or with positive language (e.g., “increase,” “up-regulated”, “activate”, “enforce,” “augment”) between the two concepts; inversely proportional to the distance (e.g., number of letters or words) between these two concepts with no negative language and/or with positive language therebetween in the resource; and proportional to a number of resources that includes both concepts with no interstitial negative language and/or with no interstitial positive language. The computer system can then combine (e.g., sum, average) the negative and positive action component to derive an (composite) action characteristic between the two concepts.

For example, the word vector cube can represent a high association score and a positive action characteristic between a first concept in a disease domain and a second concept in a gene domain. Accordingly, in this example, the first and second concepts may be frequently described together in individual resources in the corpus of resources; and presence of the disease and presence of the gene may be strongly correlated, which may indicate that the gene predicts presentation of the disease and/or the disease activates expression of the gene.

In another example, the word vector cube represents a high association score and a negative action characteristic between a first concept in the disease domain and a second concept in the bacterium domain. Accordingly, in this example, the first and second concepts are frequently described together in individual resources; and absence or mitigation of the disease and presence of the bacteria may be strongly correlated, which may indicate that the bacteria offers resistance to the disease and/or the bacteria is a prophylactic treatment for the disease.

In yet another example, the word vector cube represents a high association score and a neutral action characteristic between a first concept in the bacterium domain and a second concept in the compound domain. Accordingly, in this example, the first and second concepts are frequently described together in individual resources; but the corpus of resources are silent to or fail to return consensus on effects of the compound (e.g., second concept) on the growth of presence of the bacteria (e.g., first concept)—or vice versa.

### 6.4 Semantic Network Construction

The computer system can then: populate a semantic network with a constellation of nodes, each representing a unique concept—in the set of target domains—described in at least one resource in the corpus of resources; label each node with its corresponding domain; define connections between nodes in the semantic network; label each connection with an association score for the two concepts represented by the nodes its connects; and/or label each connection with an action characteristic derived from the word vector cube and/or interpreted directly from the corpus of resources into a semantic network.

The computer system can therefore: fuse the corpus of papers, journal publications, and patient health records into a network of language embeds (e.g., a “word vector cube”); derive association scores between concepts represented in the word vector cube; detect or predict domains of concepts in the word vector cube; derive action characteristics between concepts represented in the word vector cube; represent these concepts as nodes in the semantic network; label each node with the domain of the concept it represents; connect (or “link”) pairs of nodes according to the association scores for pairs of concepts represented by these nodes; and label connections between nodes with action characteristics and association scores for pairs of concepts represented by these nodes.

Furthermore, the computer system can: project sets of edges, between a target node and a termination node in the semantic network, onto a virtual surface to generate a visualization of a region of the semantic network representing connections between a target concept and a target domain; label the target node (e.g., start node) with the corresponding concept (e.g., target compound, target disease, target health condition, target therapy), contained in the target node, in the visualization; label nodes within the target domain with corresponding concepts (e.g., compounds, diseases, therapies) in the visualization; label edges, represented in the visualization, with action characteristics and association scores extracted from these edges; and render the visualization within the research portal for a user to interface with the visualization of the region of the semantic network to select a target concept within a target domain (e.g., target compound, target disease, target therapy).

Therefore, the computer system can generate a visualization of a selected region of the semantic network within the research portal to enable the user to review the visualization of the region of the semantic network and select a target concept from a target domain within the visualization.

### 6.5 Resource Callback

In one variation, the computer system also writes identifiers of resources that informed connections between nodes in the semantic network to these connections.

For example, for a connection between a first node containing a first concept and a second node containing a second concept, the computer system can: retrieve an identification number (e.g., “ISBN,” “ISSN,” or “DOI”), web address, or other unique identifier for each paper that contains both the first and second concepts; define an unique identifier to each medical record that contains both the first and second concepts; and write these identifiers to the connection between the first and second nodes. Later, the computer system can extract these identifiers from the semantic network, retrieve a set of resources based on these identifiers, and present these resources to the user to support a system-generated hypothesis when a user selects an edge intersecting this connection.

### 7. User Query

Generally, the computer system interfaces with a research portal (or “user portal”) to receive a set of natural language search terms entered by a user to select a target concept and/or a target domain. The set of natural language search terms can include one or more of: a particular disease, syndrome, and/or disease stage or a generic disease domain term; a particular symptom or a generic symptom domain term; a particular bacterium or a generic bacteria domain term; a particular therapy or a generic therapy domain term; a particular gene or generic gene domain term; a particular compound, pharmacologic substance, inorganic chemical, organic chemical, or generic compound domain term; a particular protein, peptide, and/or amino acid or a generic protein domain term; a particular hormone or a generic hormone domain term; a particular enzyme or a generic enzyme domain term; a particular virus or a generic virus domain term; a particular fungus or a generic fungi domain term; a particular patient population characteristic or a generic patient characteristic domain term; or a particular pathway or experiment action or a generic treatment domain term.

In one variation, the computer system can implement methods and techniques described above to project sets of edges onto a virtual surface to: generate a visualization of the semantic network representing connections between chemical and biological concepts; label sets of edges, represented in the visualization, with concepts extracted from nodes between the target node and the subset of nodes in the semantic network; render the visualization within the research portal for the user; and receive selection of the target compound, the target health condition, and the target direction within the visualization of the semantic network from the research portal (e.g., user interfaces with the visualization of the semantic network to select a target compound, a target health condition, and a target direction from the visualization).

### 8. Compound as Starting Input

Generally, the computer system can interface with the research portal to receive a query for a target compound, a target health condition, and a target direction entered by a user. The computer system can then identify edges between the target compound and the target health condition within the semantic network, extract action characteristics and patient characteristics from nodal connections and intermediate nodes along these edges, and define inclusion criteria and exclusion criteria to compile into a specification for a clinical trial.

In one implementation, the computer system can: receive a query for a target health condition, a target compound, and a target direction from a research portal; identify a first edge—including intermediate nodes representing a first set of patient characteristics and a first composite action characteristic—coupling a target node representing the target compound and a termination node representing the target health condition in a semantic network; and identify a second edge—including intermediate nodes representing a second set of patient characteristics and a second composite action characteristic—coupling the target node representing the target compound and a termination node representing the target health condition in the semantic network. Then, in response to the first composite action characteristic falling within a first range corresponding to the target direction and, in response to the second composite action characteristic falling within a second range distinct from the target direction, the computer system can: isolate a first patient characteristic, contained in the first set of patient characteristics and excluded from the second set of patient characteristics, as inclusion criteria; and define a second patient characteristic, contained in the second set of patient characteristics and excluded from first set of patient characteristics, as exclusion criteria. The computer system can then aggregate the inclusion criteria and exclusion criteria into a specification for the clinical trial.

### 8.1 Compound Selection+Target Domain

In one implementation in which the user has identified or selected a compound and is in search of an application for the compound, the user selects a “generic compound domain” within the user interface and inputs a description of the compound (i.e., a “target compound”). The user then selects a “health condition” domain to trigger the computer system to identify health condition concepts that fall near the compound in the semantic network—that is, nodes in the semantic network labeled with a health condition domain and that fall near other nodes containing the compound within the semantic network.

### 8.2 Proximal Health Conditions

In one implementation, the computer system can then query the semantic network for a set of health condition concepts (e.g., diagnoses, symptoms) proximal a node representing the target compound in the semantic network. In particular, once the user enters the target compound, the computer system queries the semantic network for an address of a target compound node within a “compound” domain and containing a language concept representing the target compound.

In one variation, the computer system scans the semantic network for a set of nodes—within a “health condition” domain—nearest the target compound node. For example, the computer system can isolate a set of health condition nodes within a threshold distance of the target compound node, such as a threshold Euclidean distance in n-dimensions of the semantic network between a health condition node and the target compound node.

In another example, the computer system can isolate a set of health condition nodes that fall within a threshold number of “hops” of the target compound node, such as health condition nodes connected to the target compound node by as few as five other intermediate nodes in any domain.

In yet another example, the computer system can isolate a target quantity of (e.g., five) health conditions nearest the target compound node (e.g., in a Euclidean distance in n-dimensions of the semantic network) or connected to the target compound node by fewest intermediate nodes.

In yet another example, the computer system can: define a radius limit for a distance from the target node representing the target compound to health condition nodes in the target domain; and identify the set of health condition nodes, in the semantic network, in the target domain and within the radius limit of the target node representing the target compound.

### 8.2.1 Isolate Nodes in Target Domain

Furthermore, for a first health condition node in this set of health condition nodes, the computer system can derive a first set of edges (each representing an action pathway) connecting the health condition node, the target compound node, and a set of (e.g., no, one, or more) intermediate nodes therebetween, such as: all permutations of discrete and partially-overlapping edges connecting the target compound and health condition nodes in the semantic network; all permutations of discrete and partially-overlapping edges connecting the target compound and health condition nodes in the semantic network up to a maximum traversal distance and/or up to a maximum number of hops between the target compound and health condition nodes; or all discrete edges of shortest traversal distance or fewest hops connecting the target compound and health condition nodes in the semantic network.

For a first edge in this first set of edges, the computer system then: reads a first set of association scores between nodes along this first edge; and calculates a first composite association score based on a combination of these association scores, such as an average of the first set of association scores, divided by a quantity of hops from the target compound node to the health condition node along the first edge.

The computer system then: repeats this process for each other edge in the first set of edges; and calculates an aggregate association score between the first health condition and the target compound node, such as based on a sum of composite association scores of these edges between the target compound and health condition nodes.

The computer system then repeats this process for each other health condition node in the set of health condition nodes.

### 8.3 Target Health Condition

In one implementation, the computer system can: sort or rank these health conditions associated with these health condition nodes based on corresponding aggregate association scores; extract health condition descriptions (or identifiers, language concepts) from these health condition nodes; present a ranked list of these health condition descriptions to the user within the research portal; and prompt the user to select a target health condition from this health condition list ranked by aggregate association scores.

In one variation, the computer system can: compile a list of health condition concepts, corresponding to these health condition nodes, ranked by composite association score; and return the list of health condition concepts, ranked by composite association score to the research portal; present this ranked list of these health condition concepts to the user within the research portal; and prompt the user to select a target health condition from this health condition list ranked by composite association scores.

Additionally or alternatively, the computer system can: render a visualization of a region of the semantic network containing these health condition and target compound nodes; highlight (e.g., color-code) or label these health condition nodes and edges connecting these health condition nodes to the target compound node—in this visualization—based on corresponding aggregate association scores (and/or composite association scores); and prompt the user to select a target health condition from this visualization.

However, the computer system can implement any other method or technique to present the set of health conditions to the user and receive selection of a target health condition from the set of health conditions from the user.

### 8.4 Target Edges

Once the computer system receives selection of a target health condition, from the set of health conditions, from the research portal, the computer system can: identify a first edge coupling a target node representing the target compound and a termination node representing the target health condition in the semantic network; calculate a first composite action characteristic for the first edge based on a first combination of action characteristics stored in connections along the first edge; extract a first set of patient characteristics from intermediate nodes, storing chemical and biological concepts, along the first edge; identify a second edge coupling the target node representing the target compound and the termination node representing the target health condition in the semantic network; calculate a second composite action characteristic for the second edge based on a second combination of action characteristics stored in connections along the second edge; and extract a second set of patient characteristics from intermediate nodes, storing chemical and biological concepts, along the second edge.

In one variation, the computer system identifies the first edge coupling the target node representing the target compound to the termination node representing the target health condition, separated by fewer than a threshold quantity of intermediate nodes in the semantic network; and identifies the second edge coupling the target node representing the target compound to the termination node, representing the target health condition, separated by fewer than the threshold quantity of intermediate nodes in the semantic network.

### 8.5 Target Direction+Action Characteristics+Association Scores

The computer system can also interface with the research portal to receive natural language search terms entered by a user to select a target direction (e.g., a positive directional keyword, a negative keyword, a value, a score) within the research portal.

In one implementation, the computer system can present a list of directional keywords within the research portal and the user can interface with the research portal to select a target direction (e.g., directional keyword) from the list of directional keywords. In this implementation, the target direction (e.g., directional keyword) can include: upregulates; downregulates; catalyzes; inhibits; starts; stops; causes; prevents; promotes; demotes; grows; kills; suppresses; yields; induces; and reduces; etc.

Furthermore, the computer system can extract a first set of action characteristics, representing directions of correlations between the target compound and the target health condition, based on the presence of directional keywords from the first edge; and transform the first set of action characteristics into a first set of values within a value range (e.g., −1.000 to +1.000), as described above. The computer system can then: calculate a first composite action characteristic for the first edge based on a combination (e.g., sum, average) of the first set of values; extract a second set of action characteristics, representing directions of correlations between the target compound and the target health condition, based on the presence of directional keywords from the second edge; transform the second set of action characteristics into a second set of values within the value range (e.g., −1.000 to +1.000); and calculate a second composite action characteristic for the second edge based on a combination (e.g., sum, average) of the second set of values.

Additionally or alternatively, the computer system can: extract a first set of association scores representing correlations between the target compound and the target health condition from the first edge; calculate a first composite association score based on a combination (e.g., sum, average) of the first set of association scores; extract a second set of association scores representing correlations between the target compound and the target health condition from the second edge; and calculate a second composite association score based on a combination (e.g., sum, average) of the second set of association scores.

The computer system can then leverage the target direction, action characteristics within a value range, and association scores exceeding a threshold score to isolate patient characteristics for a clinical trial, as further described below.

### 8.6 Clinical Trial Specification: Inclusion Criteria+Exclusion Criteria

Generally, the computer system can then execute Blocks of the method S100 to define patient characteristics as inclusion criteria and exclusion criteria based on these composite action characteristics and/or composite association scores extracted from the first edge and the second edge. The computer system can then aggregate these inclusion criteria and exclusion criteria into a specification for a clinical trial.

8.6.1 Patient Characteristics+Action Characteristics

In one implementation, the computer system can: extract a first set of patient characteristics from intermediate nodes, storing chemical and biological concepts, along the first edge; and extract a second set of patient characteristics from intermediate nodes, storing chemical and biological concepts, along the second edge. Then, in response to the first composite action characteristic falling within a first range corresponding to the target direction and in response to the second composite action characteristic falling within a second range distinct from the target direction, the computer system can: define a first patient characteristic, contained in the first set of patient characteristics and excluded from the second set of patient characteristics, as inclusion criteria; define a second patient characteristic, contained in the second set of patient characteristics and excluded from first set of patient characteristics, as exclusion criteria; and aggregate inclusion criteria and exclusion criteria into a specification for the clinical trial.

In one example, the computer system can: extract a first set of patient characteristics such as a first set of biomarkers from intermediate nodes, storing biomarker concepts, along the first edge in the semantic network; extract a second set of patient characteristics such as a second set of biomarkers from intermediate nodes, storing biomarker concepts, along the second edge in the semantic network. Then, in response to the first composite action characteristic falling within a first range (e.g., between −1.000 and 0.000) corresponding to the target direction (e.g., negative directional keyword) and in response to the second composite action characteristic falling within a second range (e.g., between 0.000 and 1.000) distinct from the target direction, the computer system can: define a first patient characteristic such as a first biomarker, contained in the first set of biomarkers and excluded from the second set of biomarkers, as inclusion criteria; and define a second patient characteristic such as a second biomarker, contained in the second set of biomarkers and excluded from first set of biomarkers, as exclusion criteria; and aggregate inclusion criteria and exclusion criteria into a specification for the clinical trial to validate efficacy of the target compound exhibiting the target direction (e.g., suppresses, catalyzes, reduces, prevents) on the target health condition for a patient population exhibiting the first biomarker.

In another example, the computer system can: extract a first set of patient characteristics such as a first set of genes from intermediate nodes, storing gene concepts, along the first edge in the semantic network; and extract a second set of patient characteristics such as a second set of genes from intermediate nodes, storing gene concepts, along the second edge in the semantic network. Then, in response to the first composite action characteristic falling within a first range (e.g., between −0.500 and 0.000) corresponding to the target direction (e.g., negative directional keyword) and in response to the second composite action characteristic falling within a second range (e.g., between 0.000 and +0.500) distinct from the target direction, the computer system can: define a first patient characteristic such as a first gene, contained in the first set of genes and excluded from the second set of genes, as inclusion criteria; define a second patient characteristic such as a second gene, contained in the second set of genes and excluded from first set of genes, as exclusion criteria; and aggregate inclusion criteria and exclusion criteria into a specification for the clinical trial to validate efficacy of the target compound exhibiting the target direction (e.g., suppresses, catalyzes, reduces, prevents) on the target health condition for a patient population exhibiting the first gene.

In yet another example, the computer system can: extract a first set of patient characteristics such as a first set of microbiomes from intermediate nodes, storing microbiome concepts, along the first edge in the semantic network; extract a second set of patient characteristics such as a second set of microbiomes from intermediate nodes, storing microbiome concepts, along the second edge in the semantic network. Then, in response to the first composite action characteristic falling within a first range (e.g., between −1.000 and −0.800) corresponding to the target direction (e.g., negative directional keyword) and in response to the second composite action characteristic falling within a second range (e.g., between 0.000 and +1.000) distinct from the target direction, the computer system can: define a first patient characteristic such as a first microbiome, contained in the first set of microbiomes and excluded from the second set of microbiomes, as inclusion criteria; define a second patient characteristic such as a second microbiome, contained in the second set of genes and excluded from the first set of genes, as exclusion criteria; and aggregate inclusion criteria and exclusion criteria into a specification for the clinical trial to validate efficacy of the target compound exhibiting the target direction (e.g., causes, induces, grows) on the target health condition for a patient population exhibiting the first microbiome.

Therefore, the computer system can extract patient characteristics from chemical and biological concepts stored in intermediate nodes along an edge coupling the target node representing the target compound and the termination node representing the target health condition. The computer system can then leverage these patient characteristics to define inclusion and exclusion criteria and compile these inclusion and exclusion criteria into a specification for a clinical trial to validate efficacy of the target compound exhibiting the target direction on the target health condition for a patient population exhibiting a particular patient characteristic (e.g., a gene, a biomarker, a microbiome, a gut bacterium).

### 8.6.2 Patient Characteristics+Association Scores

In one implementation, the computer system can execute Blocks of the method S100 to define patient characteristics as inclusion criteria and exclusion criteria based on composite action characteristics and composite association scores extracted from the first edge and the second edge.

In one variation, the computer system can: extract a first composite action characteristic and a first composite association score from connections along the first edge; extract a first set of patient characteristics from intermediate nodes, storing chemical and biological concepts, along the first edge; extract a second composite action characteristic and a second composite association score from connections along the second edge; and extract a second set of patient characteristics from intermediate nodes, storing chemical and biological concepts, along the second edge. Then, in response to the first composite action characteristic falling within a first range corresponding to the target direction and in response to the first composite association score exceeding a threshold score, the computer system can define a first patient characteristic, contained in the first set of patient characteristics and excluded from the second set of patient characteristics, as inclusion criteria. Additionally, in response to the second composite action characteristic falling within a second range distinct from the target direction and in response to the second composite association score exceeding the threshold score the computer system can define a second patient characteristic, contained in the second set of patient characteristics and excluded from first set of patient characteristics, as exclusion criteria. The computer system can then: aggregate inclusion criteria and exclusion criteria into a specification for a clinical trial; and return the specification for the clinical trial to the research portal.

In one example, the computer system can: extract a first set of patient characteristics such as a first set of gut bacteria from intermediate nodes, storing gut bacteria concepts, along the first edge in the semantic network; and extract a second set of patient characteristics such as a second set of gut bacteria from intermediate nodes, storing gut bacteria concepts, along the second edge in the semantic network. Then, in response to the first composite action characteristic falling within a first range (e.g., between 0.000 and +1.000) corresponding to the target direction (e.g., positive directional keyword) and in response to the first composite association score exceeding a threshold score (e.g., 0.40), the computer system can define a first patient characteristic such as a first gut bacterium, contained in the first set of gut bacteria and excluded from the second set of gut bacteria, as inclusion criteria. Then, in response to the second composite action characteristic falling within a second range (e.g., between −1.000 and 0.000) distinct from the target direction and in response to the second composite association score exceeding the threshold score (e.g., 0.40), the computer system can define a second patient characteristic such as a second gut bacterium, contained in the second set of gut bacteria and excluded from first set of gut bacteria, as exclusion criteria. The computer system can then aggregate inclusion criteria and exclusion criteria into a specification for the clinical trial to validate efficacy of the target compound exhibiting the target direction (e.g., upregulates, induces, demotes, grows) on the target health condition for a patient population exhibiting the first gut bacterium.

Furthermore, the computer system can predict a nominal effect of the target compound on a patient exhibiting the first gut bacterium and validate the nominal effect with the specification for the clinical trial. In particular, the computer system can: predict a nominal effect of the target compound on a patient, exhibiting the first gut bacterium, based on concepts contained in intermediate nodes along the first edge; generate a prompt for the user to approve the nominal effect of the target compound on the patient exhibiting the first gut bacterium; and serve the prompt to the user within the research portal. Then, in response to approval of the nominal effect, the computer system can aggregate inclusion criteria and exclusion criteria into the specification for the clinical trial to validate the target effect of the target compound on the patient exhibiting the first gut bacterium. Thus, the computer system can predict an effect of the target compound on a patient exhibiting a particular patient characteristic and the user can approve the effect prior to aggregating inclusion criteria and exclusion criteria into a specification for the clinical trial.

### 8.6.3 Linked Resources

In one variation, the computer system can link scientific publications containing the patient characteristic defined as inclusion criteria and/or containing the patient characteristic as exclusion criteria within the specification for the clinical trial.

For example, the computer system can: extract a first set of scientific publications containing the first patient characteristic, contained in the first set of patient characteristics, from the semantic network; extract a second set of scientific publications containing the second patient characteristic, contained in the second set of patient characteristics, from the semantic network; compile inclusion criteria linked to the first set of scientific publications and exclusion criteria linked to the second set of scientific publications into the specification for the clinical trial; and present the specification for the clinical trial within the research portal for the user. Thus, the computer system can link scientific publications to patient characteristics to enable timely review of inclusion criteria and exclusion criteria by a user within the research portal.

### 9. Variation: Compound as Input+Action Pathways

In one variation, the computer system can implement methods and techniques described above to prompt the user to enter or define a target compound. The computer system can then interface with the user and search the semantic network to: identify and select a target health condition that may affect the target compound; isolate an edge (representing a target action pathway)—corresponding to a target effect predicted by consumption of the target compound by a population of patients—defined by a series of nodes connecting the target compound and the target health condition in the semantic network; and define a set of inclusion and exclusion criteria that uniquely distinguishes the target action pathway from other action pathways connecting the target compound and the target health condition in the semantic network.

In one variation, the computer system also derives patient characteristics and/or effects predicted by each action pathway based on language concepts contained in nodes along edges between the health condition and target compound nodes in the semantic network, as described above.

### 9.1 Patient Characteristics

In one implementation, for a first action pathway between the target compound and a first health condition node, the computer system: extracts a set of language concepts from intermediate nodes and connections between nodes along a first edge of the semantic network representing the first action pathway; isolates a subset of these language concepts corresponding to patient characteristics, such as language concepts extracted from intermediate nodes—along the first edge of the semantic network—in the health condition domain; and presents this subset of language concepts as a first set of patient characteristics for this first action pathway.

For example, the computer system presents a first health condition—associated with the first action pathway—with this first set of patient characteristics. In another example, the computer system: locates a first caption block on the visualization of the semantic network proximal the first action pathway; and populates the first caption block with the first set of patient characteristics.

### 9.2 Effect

Additionally or alternatively, for the first action pathway, the computer system can predict an effect of the target compound on a patient—exhibiting the first set of patient characteristics—based on language concepts contained in nodes and connections between nodes along the first edge in the semantic network.

In one example, the first edge—representing the first action pathway—contains: a start node representing the target compound; a terminal node representing the first health condition; and an intermediate node representing a particular oral bacterium and directly connecting the start node and the terminal nodes in the semantic network. In this example, if an action characteristic between the target compound start node and the intermediate “bacterium” node is positive, the computer system can predict a direct correlation between the target compound and the particular bacterium (e.g., that the compound promotes the particular bacterium). Similarly, if an action characteristic between the intermediate “bacterium” node and the first health condition terminal node is negative, the computer system can predict an inverse correlation between the particular bacterium and the first health condition (e.g., the particular bacterium reduces frequency or severity of the first health condition).

Accordingly, the computer system can: generate a prediction for an effect of the target compound on the first health condition according to the first action pathway, such as including “the [target compound] promotes the [particular bacterium], which reduces [severity or frequency] of the health condition”; and present this effect with the first health condition to the user, such as in the form of a textual statement or by annotating connections between nodes along this first edge of the semantic network. (For example, the computer system can include a reasoning module configured to transform edges between nodes in the semantic network back into a natural language (or visual) description of the predicted mechanism of an action pathway selected by or presented to the user.) The computer system can also populate the first set of patient characteristics to include presence of the particular oral bacterium as inclusion criteria for the first action pathway.

In another example, the first edge contains: a first intermediate node representing a particular gene and directly connected to the target compound start node in the semantic network; and a second intermediate node representing a particular gastrointestinal bacterium and connecting the first intermediate node and the first health condition terminal node in the semantic network. In this example, if a first action characteristic between the target compound start node and the first intermediate “gene” node is positive, the computer system can predict a direct correlation between the target compound and the particular gene (e.g., the compound upregulates the particular gene). Similarly, if a second action characteristic between the first intermediate “gene” node and the second intermediate “bacterium” node is positive, the computer system can predict a direct correlation between the gene and the particular bacterium (e.g., expression of the gene promotes the particular bacterium). Furthermore, if a third action characteristic between the second intermediate “bacterium” node and the first health condition terminal node is negative, the computer system can predict an inverse correlation between the particular bacterium and the first health condition (e.g., presence of the bacterium suppresses the first health condition).

Accordingly, the computer system can generate a prediction for a first effect of the target compound according to the first action pathway, including “the [target compound] upregulates the [particular gene], which promotes the [particular bacterium], which reduces the first health condition.” The computer system can then return this first effect to the user. The computer system can also populate the first set of patient characteristics to include the particular gene and presence of the particular gastrointestinal bacterium as inclusion criteria for the first action pathway.

### 9.3 Resources

The computer system can also: extract identifiers of resources that informed connections between nodes along the first edge from the semantic network; and present a list of or links to these resources—with the first action pathway and the first health condition—to the user.

The computer system can repeat this process for each other action pathway in the set of action pathways connecting health condition and target compound nodes in the semantic network.

### 9.4 Target Action Pathways

The user may then select a target health condition, such as by selecting the target health condition from the list of health conditions—ranked by aggregate and/or composite association scores—or selecting a corresponding node in the visualization of the semantic network. The computer system can then: aggregate a set of action pathways—previously identified as described above—and corresponding to the set of patient characteristics, predicted effects, etc. for the target health condition; and present this set of action pathways to the user, such as in the form of a list or annotated visualization of a region of the semantic network containing corresponding edges.

Alternatively, the computer system can repeat the foregoing methods and techniques to recalculate a set of action pathways—and corresponding set of patient characteristics, predicted effects, etc.—between the target compound and target action pathway nodes, such as for all permutations of action pathways characterized by aggregate association scores greater than a threshold score and/or containing nodes spanning fewer than a threshold number of hops.

The computer system can then prompt the user to select a target action pathway from this set of action pathways.

### 9.5 Manual Inclusion/Exclusion Criteria as Semantic Network Filters

In one variation, prior to identifying a set of action pathways between the target compound and the target health condition, the computer system: prompts the user to manually enter or select inclusion and/or exclusion criteria (e.g., including or excluding patients with certain genetic markers); filters or mutes nodes of the semantic network based on these manually-entered criteria; and then identifies a set of action pathways—in the remaining semantic network—that extend between nodes representing the target compound and the target health condition.

Alternatively, the computer system can implement the foregoing methods and techniques to identify a set of action pathways between the target compound and the target health condition. The computer system can then: interface with the user to define an initial set of inclusion and/or exclusion criteria; and identify a subset of these action pathways that a) include (or fall near) nodes labeled with these inclusion criteria and b) exclude (or are far from) nodes labeled with these exclusion criteria.

For example, the computer system can: implement methods and techniques described above to extract patient characteristics from each action pathway in the set of action pathways connecting the target compound and the target health condition; present an aggregated list of these patient characteristics to the user, such as in the form of a histogram identifying each characteristic and the frequency of each characteristic across the set of action pathways; and prompt the user to manually select from these patient characteristics to populate a list of inclusion criteria and/or a list of exclusion criteria.

Then, as the user populates these lists of inclusion and exclusion criteria, the computer system can: filter the set of action pathways to include a subset of action pathways that fulfill these criteria; and present or visualize removal of action pathways from this subset and/or the action pathways that remain viable given the criteria selected by the user. For example, the computer system can render a 2D representation of the semantic network, including nodes representing the target compound, the target health condition, and action pathways extending therebetween. In this example, when the user selects a first inclusion criteria, the computer system can: identify a first subset of action pathways that exclude nodes labeled within or containing this first inclusion criteria; and remove, hide, or grey-out the first subset of action pathways in the visualization of the semantic network. Similarly, when the user selects a second exclusion criteria, the computer system can: identify a second subset of action pathways that include nodes labeled within or containing this second exclusion criteria; and remove, hide, or grey-out the second subset of action pathways in the visualization of the semantic network.

In this variation, if multiple action pathways fulfill the criteria thus manually entered or selected by the user, the computer system can implement methods and techniques described above to: present a visualization of these remaining action pathways in the semantic network; and prompt the user to select a particular action pathway from this set. Additionally or alternatively, the computer system can: generate a list of patient characteristics and predicted effects for each of these remaining action pathways; present descriptions of differences between lists of these patient characteristics and/or predicted effects to the user; and prompt the user to select a particular action pathway from this set.

Otherwise, if a single action pathway fulfills the criteria thus manually entered or selected by the user, the computer system can implement methods and techniques described above to: present a visualization of this sole remaining action pathway in the semantic network; and prompt the user to confirm this action pathway as the target action pathway.

### 9.6 Patient Characteristics for Target Action Pathway

The computer system can then: interpret a set of inclusion criteria based on the first set of patient characteristics; and interpret a set of exclusion criteria based on the second set of patient characteristics. In particular, once the user selects the target action pathway, the computer system can identify: a first set of patient characteristics that describe the target action pathway; and a second set of patient characteristics that describe other action pathways—in the set of action pathways between the target action pathway and target compound nodes in the semantic network—and that are distinct from the target action pathway.

In one implementation, for each action pathway between the target health condition and target compound nodes, the computer system can implement methods and techniques described above to extract all patient characteristics from nodes (and node connections) along the action pathway. The computer system can then then: queue a first set of patient characteristics associated with the target action pathway as possible inclusion criteria; calculate a union of all patient characteristics for all other action pathways between the target health condition and the target compound nodes; identify a second set of patient characteristics contained in the union of patient characteristics associated with these other action pathways but not contained in—or specifically in conflict with—the first set of patient characteristics associated with the target action pathway; and queue the second set of patient characteristics as possible exclusion criteria.

In one example: the target action pathway includes a node in a patient characteristic domain labeled with a first gene; and a second action pathway includes a node in the patient characteristic domain labeled with a second gene. In this example, if the first and second gene are mutually exclusive in a patient, the computer system can add the second gene to the second list of patient characteristics or directly populate the exclusion list with the second gene.

In another example: the target action pathway includes a node in a patient characteristic domain labeled with a first gastrointestinal bacterium; and a second action pathway includes a node in the patient characteristic domain labeled with a second gastrointestinal bacterium. In this example, if the first and second gastrointestinal bacterium are not mutually exclusive in a patient, the computer system can: add the first gastrointestinal bacterium to the first list of patient characteristics or directly populate the inclusion list with the first gastrointestinal bacterium; but not add the second gastrointestinal bacterium to the second list or to the exclusion list.

In another example: the target action pathway includes a node in a patient characteristic domain labeled with a first range of blood glucose levels; and a second action pathway includes a node in the patient characteristic domain labeled with a second range of blood glucose levels. In this example, if the first and second range of blood glucose levels partially overlap, the computer system can: add the first range of blood glucose levels to the first list of patient characteristics or directly populate the inclusion list with the first gastrointestinal bacterium; and add a segment of the second range of blood glucose levels outside of the first range of blood glucose levels to the second list or to the exclusion list. Alternatively, if the first and second range of blood glucose levels overlap and exhibit low association scores within their corresponding action pathways, the computer system can: exclude the first and second ranges of blood glucose levels from the first and second sets of patient characteristics; and thus exclude the first and second ranges of blood glucose levels from inclusion and exclusion criteria.

Furthermore, for each patient characteristic in the first set of patient characteristics, the computer system can score or rank the patient characteristic—for selection as an inclusion criterion—based on an association score for the patient characteristic contained along the target action pathway within the semantic network. Similarly, for each patient characteristic in the second set of patient characteristics, the computer system can score or rank the patient characteristic—for selection as an exclusion criterion—based on an association score for the patient characteristic contained along the corresponding action pathway within the semantic network.

Therefore, the computer system can isolate and rank a set of patient characteristics—contained or linked to nodes and nodal connections along the target action pathway—as possible inclusion criteria for a clinical trial to test efficacy of the target compound in treating the target health condition according to an effect predicted by the corresponding edge in the semantic network. The computer system can similarly isolate and rank a set of patient characteristics—contained or linked to nodes and nodal connections along other action pathways but not the target action pathway—as possible exclusion criteria for this clinical trial.

### 9.7 Clinical Trial Specification

The computer system can then generate a specification for a clinical trial—to validate the target effect for the target compound—based on the set of inclusion criteria and the set of exclusion criteria.

In one implementation, the computer system: presents the first set of patient characteristics—sorted by rank or score—to the user with a prompt to select or confirm these patient characteristics as inclusion criteria; and presents the second set of patient characteristics—sorted by rank or score—to the user with a prompt to select or confirm these patient characteristics as exclusion criteria.

Furthermore, the computer system can: render a visualization of the region of the semantic network depicting the target health condition node, the target compound node, and edges representing action pathways therebetween, as described above; and selectively mute, hide, or mask action pathways that include exclusion criteria confirmed by the user and that exclude inclusion criteria confirmed by the user.

Once the user confirms all or a subset of these inclusion and exclusion criteria, the computer system can compile these inclusion and exclusion criteria (and criteria manually selected by the user as described above) to generate a specification for a clinical trial.

### 9.8 Real-Time Criteria Selection

In one variation, after the user selects the target health condition and before the user selects the target action pathway, the computer system: renders a visualization of the region of the semantic network depicting the target health condition node, the target compound node, and action pathways therebetween; implements methods and techniques described above to generate a set of patient characteristics for each action pathway connecting the target health condition and the target compound nodes in the semantic network ; renders a list of these patient characteristics; and prompts the user to manually populate a list of inclusion and exclusion criteria from this list of patient characteristics, such as by dragging these patient characteristics into inclusion and exclusion list rendered near or over the region of the semantic network.

As the user adds a patient characteristic to the inclusion list, the computer system selectively mutes, hides, or masks each action pathway that excludes this patient characteristic in all nodes and nodal connections along the edge of the semantic network that represents this action pathway. Similarly, as the user adds a patient characteristic to the exclusion list, the computer system selectively mutes, hides, or masks each action pathway that includes this patient characteristic in one more nodes or nodal connections along the edge of the semantic network that represents this action pathway.

Once a single action pathway that fulfills these inclusion and exclusion criteria remains, the computer system can: prompt the user to confirm this action pathway as a target action pathway; derive a predicted effect of the target compound for the target health condition—for a patient population with the inclusion criteria and without the exclusion criteria—as described above; prompt the user to confirm the predicted effect; and then compile these data into a specification for the clinical trial.

### 9.9 Multiple Action Pathways

In one variation, the computer system similarly: cooperates with the user to select multiple (e.g., two) target action pathways; defines inclusion criteria based on the intersection of sets of patient characteristics extracted the target action pathways in this set; and defines exclusion criteria based on patient characteristics that all target action pathways in this set exclude.

### 10. Health Condition as Starting Input+Compound Domain

In one implementation, the computer system can implement methods and techniques described above to: receive selection of a target health condition from a user; query a semantic network for a set of compounds proximal the target compound; present the set of compounds to the user; receive selection of a target compound from the set of health compounds from the user; and identify a set of edges extending between a target node representing the target health condition and a termination node representing the target compound within the semantic network.

In one variation, the computer system can: receive a query for a target health condition, a target compound, and a target direction from the research portal; identify a first edge—including intermediate nodes representing a first set of patient characteristics and a first composite action characteristic—coupling a target node representing the target health condition and a termination node representing the target compound in a semantic network; and identify a second edge—including intermediate nodes representing a second set of patient characteristics and a second composite action characteristic—coupling the target node and a termination node in the semantic network. Then, in response to a first composite action characteristic falling within a first range corresponding to the target direction and in response to the second composite action characteristic falling within a second range distinct from the target direction, the computer system can: isolate a first patient characteristic, contained in the first set of patient characteristics and excluded from the second set of patient characteristics, as inclusion criteria; define a second patient characteristic, contained in the second set of patient characteristics and excluded from first set of patient characteristics, as exclusion criteria; and aggregate inclusion criteria and exclusion criteria into a specification for the clinical trial.

For example, the computer system can: receive selection of a target health condition such as a target disease at the research portal; receive selection of a target domain such as a (“generic”) compound concepts at the research portal; scan the semantic network for compound nodes representing compound concepts; and identify a set of edges coupling a target node representing the target disease to compound nodes representing compound concepts. The computer system can then: receive selection for the target compound such as a target therapy; isolate a first edge from the set of edges coupling the target node representing the target disease and the termination node representing the target therapy in the semantic network; isolate a second edge from the set of edges coupling the target node representing the target disease and the termination node representing the target therapy in the semantic network; and implement methods and techniques described above to aggregate inclusion criteria and exclusion criteria into the specification for the clinical trial to validate efficacy of the target therapy on the target disease.

### 10.1 Variation: Action Pathways

In one variation, the computer system then implements methods and techniques similar to those described above to: receive selection of a target health condition from a user; query a semantic network for a set of compounds proximal the target compound; present the set of compounds to the user; receive selection of a target compound from the set of health compounds from the user; and identify a set of action pathways extending between a node representing the target health condition and a node representing the target compound within the semantic network.

In this variation, for each action pathway in the set of action pathways, the computer system then: aggregates a set of patient characteristics from language concepts (e.g., patient traits, demographics, symptoms) contained in a series of nodes along (and near) the action pathway; and predicts an effect of the target compound on a patient, exhibiting the set of patient characteristics, based on language concepts contained in the series of nodes.

In this variation, the computer system then: prompts the user to select a target action pathway, associated with a target effect, from the set of action pathways; identifies a first set of patient characteristics that describe the target action pathway; identifies a second set of patient characteristics that describe other action pathways in the set of action pathways and that are distinct from the target action pathway; interprets a set of inclusion criteria based on the first set of patient characteristics; interprets a set of exclusion criteria based on the second set of patient characteristics; and generates a specification for a clinical trial—to validate the target effect for the target compound—based on the set of inclusion criteria and the set of exclusion criteria.

Therefore, in this variation, the computer system can implement methods and techniques similar to those described above to generate a specification for a clinical trial based on a target health condition first selected by the user—followed by selection of a target compound and then inclusion and exclusion criteria.

### 11. Patient Characteristics as Starting Input

In one variation, the computer system implements methods and techniques similar to those described above to: receive selection of a first set of patient characteristics—as an initial set of inclusion and/or exclusion criteria—from a user; and query a semantic network for a set of clusters of nodes containing language concepts approximating the first set of patient characteristics.

In this variation, for each cluster of nodes in the set of clusters of nodes, the computer system then: identifies a first node representing the health condition and a second node representing the compound within the semantic network proximal the cluster of nodes in the semantic network; and identifies a set of action pathways extending between the first node and the second node within the semantic network. For each action pathway in the set of action pathways, the computer system aggregates a set of patient characteristics from language concepts (e.g., patient traits, demographics, symptoms) contained in a series of nodes along (and near) the action pathway; and predicts an effect of the compound on a patient, exhibiting the set of patient characteristics, based on language concepts contained in the series of nodes.

The computer system then: prompts the user to select a target action pathway, associated with a target effect, from a set of action pathways associated with a cluster of nodes in the set of nodes; extracts a second set of patient characteristics that describe the target action pathway; interprets a set of inclusion and exclusion criteria based on the first set of patient characteristics and the second set of patient characteristics; and generates a specification for a clinical trial—to validate the target effect for the target compound on the target health condition—based on the set of inclusion criteria and the set of exclusion criteria.

Therefore, in this variation, the computer system can implement methods and techniques similar to those described above to generate a specification for a clinical trial based on patient characteristics first selected by the user—followed by selection of a target health condition and a target compound.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method for prescribing a clinical trial comprising:
receiving a query for a target compound, a target health condition, and a target direction from a research portal;
identifying a first edge coupling a target node representing the target compound and a termination node representing the target health condition in a semantic network;
calculating a first composite action characteristic for the first edge based on a first combination of action characteristics stored in connections along the first edge;
extracting a first set of patient characteristics from intermediate nodes, storing chemical and biological concepts, along the first edge;
identifying a second edge coupling the target node representing the target compound and the termination node representing the target health condition in the semantic network;
calculating a second composite action characteristic for the second edge based on a second combination of action characteristics stored in connections along the second edge;
extracting a second set of patient characteristics from intermediate nodes, storing chemical and biological concepts, along the second edge;
in response to the first composite action characteristic falling within a first range corresponding to the target direction and in response to the second composite action characteristic falling within a second range distinct from the target direction:
defining a first patient characteristic, contained in the first set of patient characteristics and excluded from the second set of patient characteristics, as inclusion criteria; and
defining a second patient characteristic, contained in the second set of patient characteristics and excluded from the first set of patient characteristics, as exclusion criteria; and
aggregating inclusion criteria and exclusion criteria into a specification for the clinical trial.

2. The method of claim 1, further comprising:
accessing a corpus of scientific publications;
compiling a population of semantic concepts represented in the corpus of scientific publications into a vector space model based on:
proximity of semantic concepts within individual scientific publications, in the corpus of scientific publications; and
frequency of semantic concepts across the corpus of scientific publications;
deriving domains of a set of chemical and biological concepts in the vector space model based on proximity to domain descriptors in the vector space model;
deriving association scores between connected chemical and biological concepts, in the set of chemical and biological concepts, based on proximity in the vector space model;
deriving action characteristics between connected chemical and biological concepts, in the set of chemical and biological concepts, based on action descriptors in the vector space model; and
generating a semantic network comprising:
a set of nodes representing the set of chemical and biological concepts labeled with domains; and
a set of edges connecting the set of nodes and storing association scores and action characteristics.

3. The method of claim 2:
further comprising:
projecting the set of edges onto a virtual surface to generate a visualization of the semantic network representing connections between chemical and biological concepts;
labeling the set of edges, represented in the visualization, with concepts extracted from nodes between the target node and the subset of nodes in the semantic network; and
rendering the visualization within the research portal for the user; and
wherein receiving the query for the target compound, the target health condition, and the target direction comprises receiving selection of the target compound, the target health condition, and the target direction within the visualization of the semantic network from the research portal.

4. The method of claim 2:
further comprising extracting a first set of scientific publications containing the first patient characteristic, contained in the first set of patient characteristics, from the semantic network;
further comprising extracting a second set of scientific publications containing the second patient characteristic, contained in the second set of patient characteristics, from the semantic network;
wherein aggregating inclusion criteria and exclusion criteria into the specification for the clinical trial comprises compiling inclusion criteria linked to the first set of scientific publications and exclusion criteria linked to the second set of scientific publications into the specification for the clinical trial; and
further comprising presenting the specification for the clinical trial within the research portal for the user.

5. The method of claim 1:
wherein identifying the first edge comprises identifying the first edge coupling the target node representing the target compound to the termination node representing the target health condition, separated by fewer than a threshold quantity of intermediate nodes in the semantic network; and
wherein identifying the second edge comprises identifying the second edge coupling the target node representing the target compound to the termination node, representing the target health condition, separated by fewer than the threshold quantity of intermediate nodes in the semantic network.

6. The method of claim 1:
wherein extracting the first set of patient characteristics from intermediate nodes along the first edge comprises extracting the first set of patient characteristics comprising a first set of biomarkers from intermediate nodes, storing biomarker concepts, along the first edge;
wherein extracting the second set of patient characteristics from intermediate nodes along the second edge comprises extracting the second set of patient characteristics comprising a second set of biomarkers from intermediate nodes, storing biomarker concepts, along the second edge; and
wherein aggregating inclusion criteria and exclusion criteria comprises aggregating inclusion criteria and exclusion criteria into the specification for the clinical trial to validate efficacy of the target compound on the target health condition for a patient population exhibiting the first set of biomarkers.

7. The method of claim 1:
further comprising:
receiving selection of a target compound at the research portal;
receiving selection of a target domain comprising health condition concepts at the research portal;
scanning the semantic network for a set of health condition nodes representing health condition concepts; and
identifying a set of edges coupling the target node representing the target compound to the set of health condition nodes, representing health condition concepts;
wherein receiving the query for the target compound, the target health condition, and the target direction at the research portal comprises receiving selection for the target health condition comprising a target disease;
wherein identifying the first edge comprises isolating the first edge from the set of edges coupling the target node representing the target compound and the termination node representing the target disease in the semantic network;
wherein identifying the second edge comprises isolating the second edge from the set of edges coupling the target node representing the target compound and the termination node representing the target disease in the semantic network; and
wherein aggregating inclusion criteria and exclusion criteria comprises aggregating inclusion criteria and exclusion criteria into the specification for the clinical trial to validate efficacy of the target compound on the target disease.

8. The method of claim 1:
wherein extracting the first set of patient characteristics from intermediate nodes along the first edge comprises extracting the first set of patient characteristics comprising a first set of genes from intermediate nodes, storing gene concepts, along the first edge;
wherein extracting the second set of patient characteristics from intermediate nodes along the second edge comprises extracting the second set of patient characteristics comprising a second set of genes from intermediate nodes, storing gene concepts, along the second edge; and
wherein aggregating inclusion criteria and exclusion criteria comprises aggregating inclusion criteria and exclusion criteria into the specification for the clinical trial to validate efficacy of the target compound on the target health condition for a patient population exhibiting the first set of genes.

9. The method of claim 1:
wherein extracting the first set of patient characteristics from intermediate nodes along the first edge comprises extracting the first set of patient characteristics comprising a first set of microbiomes from intermediate nodes, storing microbiome concepts, along the first edge;
wherein extracting the second set of patient characteristics from intermediate nodes along the second edge comprises extracting the second set of patient characteristics comprising a second set of microbiomes from intermediate nodes, storing microbiome concepts, along the second edge; and
wherein aggregating inclusion criteria and exclusion criteria comprises aggregating inclusion criteria and exclusion criteria into the specification for the clinical trial to validate efficacy of the target compound on the target health condition for a patient population exhibiting the first set of microbiomes.

10. The method of claim 1:
further comprising:
receiving selection of a target health condition comprising a target disease at the research portal;
receiving selection of a target domain comprising compound concepts at the research portal;
scanning the semantic network for compound nodes representing compound concepts; and
identifying a set of edges coupling a secondary target node representing the target disease to compound nodes representing compound concepts;
wherein receiving the query for the target compound, the target health condition, and the target direction at the research portal comprises receiving selection for the target compound comprising a target therapy;
wherein identifying the first edge comprises isolating the first edge from the set of edges coupling the secondary target node representing the target disease and a secondary termination node representing the target therapy in the semantic network;
wherein identifying the second edge comprises isolating the second edge from the set of edges coupling the secondary target node representing the target disease and the secondary termination node representing the target therapy in the semantic network; and
wherein aggregating inclusion criteria and exclusion criteria comprises aggregating inclusion criteria and exclusion criteria into the specification for the clinical trial to validate efficacy of the target therapy on the target disease.

1. method of claim 1:
further comprising:
extracting a first set of action characteristics, representing directions of correlations between the target compound and the target health condition, based on the presence of directional keywords from the first edge; and
transforming the first set of action characteristics into a first set of values within a value range;
wherein calculating the first composite action characteristic for the first edge comprises calculating the first composite action characteristic for the first edge based on a third combination of the first set of values;
further comprising:
extracting a second set of action characteristics, representing directions of correlations between the target compound and the target health condition, based on the presence of directional keywords from the second edge; and
transforming the second set of action characteristics into a second set of values within the value range; and
wherein calculating the second composite action characteristic for the second edge comprises calculating a second composite action characteristic for the second edge based on a fourth combination of the second set of values.

12. The method of claim 1:
wherein receiving the query for the target compound, the target health condition, and the target direction further comprises receiving selection of a directional keyword from the research portal;
further comprising in response to the first composite action characteristic falling within the first range corresponding to the directional keyword and in response to the second composite action characteristic falling within the second range distinct from the directional keyword:
defining a third patient characteristic, contained in the first set of patient characteristics and excluded from the second set of patient characteristics, as inclusion criteria; and
defining a fourth patient characteristic, contained in the second set of patient characteristics and excluded from the first set of patient characteristics, as exclusion criteria; and
further comprising updating inclusion criteria and exclusion criteria in the specification for the clinical trial.

13. The method of claim 12, wherein receiving the query for the target compound, the target health condition, and the target direction comprises receiving selection of the directional keyword from the research portal, the directional keyword selected from a group comprising:
upregulates;
downregulates;
catalyzes;
inhibits;
starts;
stops;
causes;
prevents;
promotes;
demotes;
grows;
kills;
suppresses;
yields;
induces; and
reduces.

14. The method of claim 1:
further comprising:
receiving selection of a target compound at the research portal;
receiving selection of a target domain comprising health condition concepts at the research portal;
scanning the semantic network for a set of health condition nodes representing health condition concepts;
identifying a set of edges coupling the target node representing the target compound to the set of health condition nodes representing health condition concepts;
for each edge in the set of edges:
extracting a set of association scores from the edge; and
calculating a composite association score based on a third combination of the set of association scores;
compiling a list of health condition concepts, corresponding to the set of health condition nodes, ranked by composite association score; and
returning the list of health condition concepts, ranked by composite association score to the research portal; and
wherein receiving the query for the target compound, the target health condition, and the target direction at the research portal comprises receiving selection for the target health condition from the list of health condition concepts.

15. The method of claim 14:
further comprising, defining a radius limit for a distance from the target node representing the target compound to health condition nodes in the target domain; and
wherein scanning the semantic network for the set of health condition nodes comprises identifying the set of health condition nodes, in the semantic network, in the target domain and within the radius limit of the target node representing the target compound.

16. method of claim 1, further comprising:
extracting a first set of association scores representing correlations between the target compound and the target health condition from the first edge;
calculating a first composite association score based on a third combination of the first set of association scores;
extracting a second set of association scores representing correlations between the target compound and the target health condition from the second edge;
calculating a second composite association score based on a fourth combination of the second set of association scores;
in response to the first composite action characteristic falling within the first range corresponding to the target direction and in response to the first composite association score exceeding a threshold score:
defining a third patient characteristic, contained in the first set of patient characteristics and excluded from the second set of patient characteristics, as inclusion criteria; and
in response to the second composite action characteristic falling within a second range distinct from the target direction and in response to the second composite association score exceeding the threshold score:
defining a fourth patient characteristic, contained in the second set of patient characteristics and excluded from first set of patient characteristics, as exclusion criteria; and
updating inclusion criteria and exclusion criteria in the specification for the clinical trial.

17. A method for prescribing a clinical trial comprising:
receiving a query for a target compound, a target health condition, and a target direction from a research portal;
identifying a first edge coupling a target node representing the target compound and a termination node representing the target health condition in a semantic network;
extracting a first composite action characteristic and a first composite association score from connections along the first edge;
extracting a first set of patient characteristics from intermediate nodes, storing chemical and biological concepts, along the first edge;
identifying a second edge coupling the target node representing the target compound and the termination node representing the target health condition in the semantic network;
extracting a second composite action characteristic and a second composite association score from connections along the second edge;
extracting a second set of patient characteristics from intermediate nodes, storing chemical and biological concepts, along the second edge;
in response to the first composite action characteristic falling within a first range corresponding to the target direction and in response to the first composite association score exceeding a threshold score:
defining a first patient characteristic, contained in the first set of patient characteristics and excluded from the second set of patient characteristics, as inclusion criteria;
in response to the second composite action characteristic falling within a second range distinct from the target direction and in response to the second composite association score exceeding the threshold score:
defining a second patient characteristic, contained in the second set of patient characteristics and excluded from first set of patient characteristics, as exclusion criteria;
aggregating inclusion criteria and exclusion criteria into a specification for a clinical trial; and
returning the specification for the clinical trial to the research portal.

18. method of claim 1:
wherein extracting the first set of patient characteristics from intermediate nodes along the first edge comprises extracting the first set of patient characteristics comprising a first set of gut bacteria from intermediate nodes, storing gut bacteria concepts, along the first edge;
wherein extracting the second set of patient characteristics from intermediate nodes along the second edge comprises extracting the second set of patient characteristics comprising a second set of gut bacteria from intermediate nodes, storing gut bacteria concepts, along the second edge; and
wherein aggregating inclusion criteria and exclusion criteria comprises aggregating inclusion criteria and exclusion criteria into the specification for the clinical trial to validate efficacy of the target compound on the target health condition for a patient population exhibiting the first set of gut bacteria.

19. The method of claim 18:
wherein defining the first patient characteristic, contained in the first set of patient characteristics comprises defining a first patient characteristic comprising a first gut bacterium, contained in the first set of gut bacteria as inclusion criteria;
further comprising:
predicting a nominal effect of the target compound on a patient, exhibiting the first gut bacterium, based on concepts contained in intermediate nodes along the first edge;
generating a prompt for the user to approve the nominal effect of the target compound on the patient exhibiting the first gut bacterium; and
serving the prompt to the user within the research portal; and
wherein aggregating inclusion criteria and exclusion criteria comprises in response to approval of the nominal effect, aggregating inclusion criteria and exclusion criteria into the specification for the clinical trial to validate the nominal effect of the target compound on the patient exhibiting the first gut bacterium.

20. A method for prescribing a clinical trial comprising:
receiving a query for a target health condition, a target compound and a target direction from a research portal;
identifying a first edge coupling a target node representing the target health condition and a termination node representing the target compound in a semantic network, the first edge comprising:
intermediate nodes representing a first set of patient characteristics; and
a first composite action characteristic;
identifying a second edge coupling the target node and a termination node in the semantic network, the second edge comprising:
intermediate nodes representing a second set of patient characteristics; and
a second composite action characteristic;
in response to the first composite action characteristic falling within a first range corresponding to the target direction and in response to the second composite action characteristic falling within a second range distinct from the target direction:
isolating a first patient characteristic, contained in the first set of patient characteristics and excluded from the second set of patient characteristics, as inclusion criteria; and
defining a second patient characteristic, contained in the second set of patient characteristics and excluded from first set of patient characteristics, as exclusion criteria; and
aggregating inclusion criteria and exclusion criteria into a specification for the clinical trial.
